(19) United States
(12) Patent Application Publication
(10) Pub. No.: US 2023/0140407 A1
(43) Pub. Date: May 4, 2023

(54) DATA AND POWER TRANSFER DEVICES FOR USE WITH MEDICAL DEVICES AND RELATED METHODS

(71) Applicant: Xenter, Inc., Salt Lake City, UT (US)

(72) Inventors: John Samawi, Los Gatos, CA (US); Raleigh Purtzer, San Clemente, CA (US); Gary J. Baldwin, Seabrook, CA (US); Nathan Knighton, Centerville, UT (US); John R. Durham, Cottonwood Heights, UT (US); Peter Liao, Santa Rosa, CA (US); Richard J. Linder, Sandy, UT (US)

(21) Appl. No.: 17/979,629

(22) Filed: Nov. 2, 2022

Related U.S. Application Data
(60) Provisional application No. 63/275,504, filed on Nov. 4, 2021.

(51) Int. Cl. A61M 39/06; A61B 5/0215

(52) U.S. Cl. CPC A61M 39/06; A61B 5/0215; A61M 2039/0258

(57) ABSTRACT

The present disclosure provides various medical devices, electronic devices, systems, and related methods. In various embodiments, an electronic device may be selectively coupled to, and removable from, a medical device. The coupling may include physical coupling, electrical coupling, or both. In one embodiment, an electronic device is configured for coupling with a hemostasis valve. The electronic device may provide power and/or data connections for an elongated flexible instrument passing through the hemostasis valve, for a sensor associated with the valve, or both. In another embodiment, the electronic device may be selectively and directly coupled with an elongated flexible instrument for power and/or communication with associated electrical components.

### CROSS-REFERENCE TO RELATED APPLICATIONS

The present application claims benefit of U.S. Provisional Patent Application No. 63/275,504, entitled “Data and Power Transfer Devices for use with Medical Devices and Related Methods”, filed on Nov. 4, 2021, the disclosure of which is incorporated by reference herein in its entirety.

### BACKGROUND

The present disclosure relates generally to medical devices and the transfer of data to or from the medical devices or among individual components of the medical device. Some non-limiting examples include intraluminal devices, such as needles, guidewires and catheters, which have various sensors for detecting or measuring one or more physiological parameters.

Guidewire devices are often used to lead or guide catheters or other interventional devices to a targeted anatomical location within a patient's body. Typically, guidewires are passed into and through a patient's vasculature in order to reach the target location, which may be, for example, at or near the patient's heart or brain. Radiographic imaging is typically utilized to assist in navigating a guidewire to the targeted location. Guidewires are available with various outer diameter sizes. Widely utilized sizes include 0.010, 0.014, 0.016, 0.018, 0.024, and 0.035 inches in diameter, for example, though they may also be smaller or larger in diameter.

In many instances, a guidewire is placed within the body during an interventional procedure where it can be used to guide multiple catheters or other interventional devices to the targeted anatomical location. Once in place, a catheter can be used, for example, to aspirate clots or other occlusions, or to deliver drugs, stents, embolic devices, radiopaque dyes, or other devices or substances for treating the patient.

These types of interventional devices can include sensors located at a distal end of the device in order to provide added functionality to the device. For example, intravascular ultrasound (IVUS) is an imaging technique that utilizes a catheter (or other elongate flexible device) with one or more ultrasound imaging sensors attached to a distal end of the catheter. Ultrasound is utilized to provide images of the targeted vasculature (e.g., the coronary arteries).

There are several challenges associated with using sensors with intraluminal devices. For example, such interventional devices have very limited space to work in, given the stringent dimensional constraints involved. Moreover, integrating the sensors with the interventional device in a way that maintains effective functionality can be challenging. Further, it can be difficult to provide adequate power and facilitate data transmission to and from the sensors or other electrical components while still maintaining the physical and operative performance of the device.

The use of such interventional devices is also challenging due to the need to manage several long lengths of wires and other components, including guidewires, power cables, data wires, and the like. Care must be taken with respect to what is allowed in the sterile field and how it is handled once within the sterile field. Additional staff is often required simply to manage such wires and cables during an interventional or other medical procedure.

As such, there is an ongoing need for improved medical devices that effectively integrate sensors and can help manage data and power in a more efficient manner and/or provide data not previously obtainable in a practical manner.

### SUMMARY

The present disclosure provides various medical devices, electronic devices, systems, and related methods. In various embodiments, an electronic device may be selectively coupled to, and removable from, a medical device. The coupling may include physical coupling, magnetic coupling, electrical coupling, or both.

In one particular embodiment, a medical device system comprises a hemostasis valve having a body defining a passage and having at least one electronic component. The system additionally includes an elongated, flexible member configured to be displaced through the passage of the hemostasis valve, wherein the elongated, flexible member includes at least one sensor. An electronic device is removably coupled with the hemostasis valve and is in electrical communication with at least one of the at least one electronic component and the at least one sensor when coupled with the hemostasis valve.

In one embodiment, the at least one sensor includes at least one of a pressure sensor, a temperature sensor, a flow sensor, and an imaging sensor.

In one embodiment, the elongated, flexible member includes a guidewire or a catheter.

In one embodiment, the electronic device includes a control unit having a processor, a power source, and a transmitting/receiving device.

In one embodiment, the electronic component includes at least one other sensor.

In one embodiment, the at least one other sensor comprises a pressure sensor.

In one embodiment, the electronic device is configured to communicate wirelessly with an external device.

In another embodiment, a medical device is provided. The medical device includes a body defining a passage therethrough, a first connector at a first end of the body, a valve at a second end of the body, wherein the body is configured to receive an elongated, flexible member through the valve and through the passage, and at least one sensor configured to measure a parameter of a fluid contained within the body.

In one embodiment, the at least one sensor includes a pressure sensor.

The in one embodiment, the body is configured for physical, magnetic, and/or electrical coupling with an electronic device.

In one embodiment, the first connector is configured as a Luer lock.

In one embodiment, the medical device further comprises at least one port in fluid communication with the passage.

In accordance with another embodiment, an electronic device is provided that comprises a control unit comprising a body. The control unit includes a processor, a device for wirelessly transmitting and receiving data, and a power source disposed in the housing. The body is configured for selectively coupling with, and removal from, a hemostasis valve.

In one embodiment, the electronic device further comprising at least one input device and at least one output device.

In one embodiment, the at least one output device includes at least one light.

In one embodiment, the at least one output device includes a plurality of lights, each light of the plurality configured to provide a status indication for an associated sensor of a medical device system.

In one embodiment, the at least one output device is configured to provide at least two status indicators.

In one embodiment, the at least two status indicators include at least two different colors.

In one embodiment, the at least one output device includes a display screen.

In one embodiment, the body is configured for selectively coupling with, and removal from, a flexible, elongated member configured for insertion into a lumen of a patient.

In accordance with another embodiment of the present disclosure, a medical device is provided that comprises a catheter having at least one array of ultrasound transducers. An electronic device is coupled to a proximal portion of the catheter, wherein the electronic device includes a control unit in communication with the at least one array of ultrasonic transducers. The control unit includes a processor, a power source, and a transmitting/receiving device configured to wirelessly communicate with an external device.

In one embodiment, the electronic device includes a housing configured as a handle to be grasped by a hand of a user.

In one embodiment, the power source includes a battery.

Various aspects, components, features, and structures of one disclosed embodiment may be combined, associated with or incorporated into other described embodiments without limitation.

### BRIEF DESCRIPTION OF THE SEVERAL VIEWS OF THE DRAWINGS

The foregoing and other advantages of the invention will become apparent upon reading the following detailed description and upon reference to the drawings in which:

FIG. 1 illustrates a medical device system according to an embodiment of the present disclosure;

FIG. 2 is a side view a component of the medical device system shown in FIG. 1 according to one embodiment of the present disclosure;

FIG. 3 is a partially exploded, perspective view of component shown in FIG. 2 according to an embodiment of the present disclosure;

FIGS. 4-8 show different configurations of a medical device system according to various embodiments of the present disclosure;

FIG. 9 illustrates a portion of the medical device system of FIG. 1 according to an embodiment of the present disclosure;

FIG. 10 illustrates a portion of the medical device system shown in FIG. 1 according to another embodiment of the present disclosure; and

FIG. 11 illustrates a medical device system according to another embodiment of the present disclosure.

### DETAILED DESCRIPTION OF THE INVENTION

Various embodiments described herein are directed toward the transfer of power and/or data to and/or from medical devices or electrical components thereof. In some embodiments, devices associated with cardiovascular, neurovascular and endovascular procedures are provided having sensors integrated therewith. For example, needles, guidewires or catheters may include sensors integrated into the structure for detecting or measuring physiological data (e.g., pressure, flow rate, pulse wave velocity, etc.), providing imaging data—often providing that data in real time during an associated procedure—or treating a condition of a patient.

In some embodiments, other sensors or electronic elements are associated with the device. For example, sensors configured to detect the presence of biological components may be incorporated into or otherwise associated with the device.

Some non-limiting examples of medical devices that may incorporate such sensors include those described in the following publications: U.S. Patent Publication No. US20210290198 (application Ser. No. 17/205,964), entitled “Guidewire for Imaging and Measurement of Pressure and Other Physiological Parameters” and filed on Mar. 18, 2021; U.S. Patent Publication No. US20210290164 (application Ser. No. 17/205,854), entitled “Catheter for Imaging and Measurement of Pressure and Other Physiological Parameters” and filed on Mar. 19, 2021; U.S. Patent Publication No. US20210290100 (application Ser. No. 17/205,754), entitled “Operatively Coupled Data and Power Transfer Device for Medical Guidewires and Catheters with Sensor” and filed on Mar. 18, 2021; and U.S. Patent Publication No. US20210290059 (application Ser. No. 17/205,614), entitled “Signal Conducting Device for Concurrent Power and Data Transfer to and From Un-wired Sensors Attached to a Medical Device” and filed on Mar. 18, 2021, the disclosures of which are each incorporated by reference herein in their entireties.

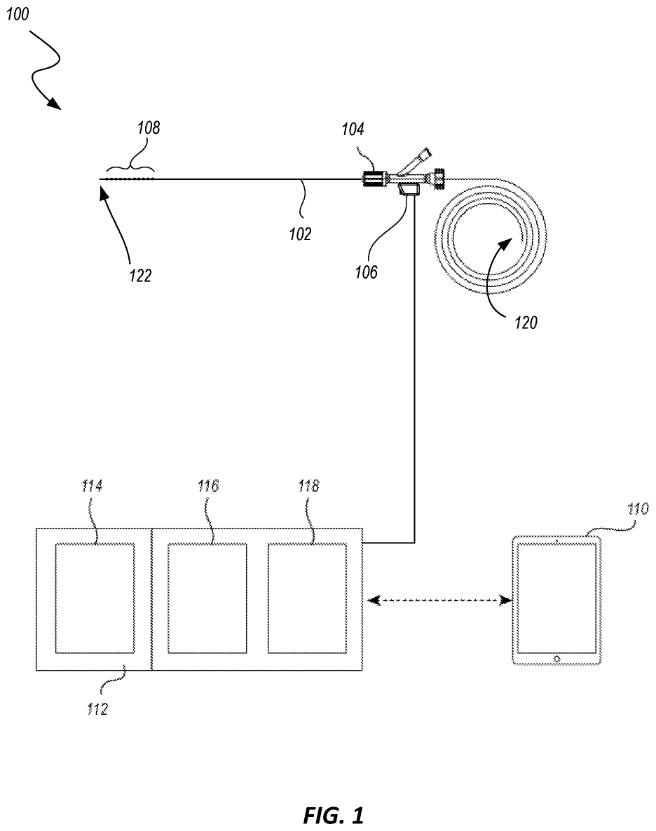

Referring to FIG. 1, a medical device system 100 is illustrated according to an embodiment of the present disclosure. As shown, the system 100 includes an intraluminal device 102 which may be configured as an elongated member (e.g., a needle, a guidewire, sheath, or a catheter), a hemostasis valve 104 (e.g., a rotating hemostasis valve (RHV) such as a Touhy valve), a data/power transfer device 106 (which may be referred to herein as an electronic device for sake of simplicity) positioned at a proximal end of the intraluminal member 102. One or more sensors 108 may be positioned at a distal end of the intraluminal member 102 and placed in communication with the electronic device 106. The electronic device 106 may include a control unit 112 (shown enlarged and in schematic form) that includes a power source 114 such as a battery, data signal processor 116, and a transmitter/receiver (referred to herein as a transceiver 118) capable of bi-directional communication. The electronic device 106 may be in communication with an external device 110 (e.g., a stationary or handheld display, tablet computer, or other input and/or output device). In one embodiment, the data transfer device 106 is in wireless communication with the external device 110 (e.g., via the transceiver 118). For example, the transceiver may include components for transmitting and receiving data using Bluetooth, ultrawideband, Wi-Fi, WiMAX, Zigbee or other such wireless technologies. The control unit may include other components (memory, circuitry, etc.) for carrying out its function of transmitting and processing electrical signals (data and power) in association with operation of the sensors 108 of the intraluminal device 102, conditioning data signals received from the sensors, if necessary, and transmitting the data or information to the external device 110. The control unit 112 may also receive instructions from the external device 110 and carry out the instructions through operation of the sensors 108 or other components of the intraluminal device 102, hemostasis valve 104 or electronic device 106.

In one embodiment, the hemostasis valve 104 and the electronic device 106 may be cooperatively configured for electrical coupling with the intraluminal device 102 to provide power to sensors 108 or other electronics associated with the intraluminal device 102 and to send and/or receive various signals provided by the sensors 108 or other electrical components.

In one embodiment, such electrical coupling may be accomplished via conductive connection. In another embodiment, such electrical coupling may be accomplished via an electrical or electromagnetic field (e.g., through a capacitive or inductive coupling).

The data signal processor 116 may be configured to receive sensor data signals, sent through the intraluminal device 102, from one or more sensors 108 (which may be located and oriented in specific patterns as individual sensors, or may be configured as identifiable sensor arrays) associated with the intraluminal device 102. The power source 114 may be configured to transmit power through the intraluminal device 102 to power the one or more sensors 108 and/or other components of the intraluminal device 102. The power source 114 may include an on-board power source, such as a battery, a battery pack, or other temporary energy source (single use or rechargeable) and/or may include a wired connection to an outside power source.

The intraluminal device 102 has a proximal end 120 and a distal end 122. The one or more sensors 108 may be located at any suitable position on the intraluminal device 102, but will typically be disposed at or near the distal end 122 which is expected to reach the targeted anatomy. As used herein, the “distal section” or “distal portion” refers to the distal-most 30 cm of the device, the distal-most 20 cm of the device, the distal-most 15 cm of the device, the distal-most 10 cm of the device, or to a range using any two of the foregoing values as endpoints. In some embodiments, the “intermediate section” may be considered as roughly the middle third of the device, and the “proximal section” or “proximal portion” may be considered as roughly the proximal third of the device.

In one embodiment, the system 100 is configured to send power to the sensors 108 through individual traces or other electrical conductors, and data signals from the sensors may be transmitted via separate transmission structures or schemes. For example, data may be transmitted by discrete traces or electrical conductors, by optical transmission, or by wireless transmission. Such transmission structures (e.g., traces) may be formed on/in, integrated with the intraluminal device 102 or formed as discrete members extending a length (or at least partial length) of the intraluminal device 102.

In another embodiment, the system 100 is configured to send power and data signals through the actual intraluminal device 102 itself rather than through traces or through separate, discrete electrical conductors. For example, in some embodiments, the intraluminal device may be configured as a guidewire wherein a structural component of the guidewire (e.g., a “core wire”, a hypo tube, or some other structural element that serves to provide or enable performance characteristics such as torquing, bending, pushing, tracking, etc.) facilitates transmission of power, data, or both types of signals. In some embodiments, multiple power and/or data signals (e.g., data signals from multiple sensors) can be sent through the intraluminal device 102 simultaneously (i.e., through a common conductor or transmission structure simultaneously). Power and/or data signals can also be sent in a “continuous” fashion. That is, the power and/or data signals can have a sufficiently high sampling rate such that the information is provided to the user within time frames that are practically “real-time” in either an analog or digital data format. Nonlimiting examples of managing power and/or data signals, along with associated components and systems, as set forth in the previously incorporated U.S. patent applications.

The intraluminal device 102 of the system 100 is configured for insertion into the body of a subject. The subject is typically a human, but in other implementations the system may be used with a non-human mammal or even non-mammalian animal. Any suitable route of administration may be utilized, depending on particular preferences and/or application needs. Common routes include femoral, radial, and jugular, but the system 100 may utilize other access routes as needed. In some embodiments, while referred to as an “intraluminal” device, the device may be used in the diagnosis or treatment of pulmonary, gastrointestinal, or other physiological systems.

The length of the intraluminal device 102 may vary according to particular application and targeted anatomical area. As an example, with the intraluminal device 102 being configured as a guidewire, the intraluminal device may have an overall length from proximal end 120 to distal end 122 of about 50 cm to about 350 cm, more commonly about 200 cm, depending on particular application needs and/or particular anatomical targets. Additionally, when configured as a guidewire, the intraluminal device 102 may exhibit an outer diameter (e.g., after application of other outer members or coatings) of about 0.008 inches to about 0.040 inches, though larger or smaller sizes may also be utilized depending on particular application needs. For example, particular embodiments may have outer diameter sizes corresponding to standard guidewire sizes such as 0.010 inches, 0.014 inches, 0.016 inches, 0.018 inches, 0.024 inches, 0.035 inches, 0.038 inches, or other such sizes common to guidewire devices. The intraluminal device 102 may be formed from stainless steel or other metal or alloy having similar appropriate properties.

The one or more sensors 108 of the system 100 may include a pressure sensor, a flow sensor, an imaging sensor, temperature sensors, a component detection sensor, or combinations thereof, for example. A pressure sensor may be sized and configured to sense a pressure, and/or changes in pressure, in the environment in which the sensor will be disposed. A flow sensor may be sized and configured to sense the fluid flow, such as velocity or other flow characteristics. A detection sensor may detect a proximity or distance to one or more detection nodes positioned external relative to the body. An imaging sensor may include any of a variety of sensor types (e.g., ultrasound transducers, optical sensors, etc.) to gather various forms of imaging data.

The one or more sensors may additionally, or alternatively, be configured to sense the presence of biological components or measure physiological parameters in the targeted anatomical location (e.g., in the blood). Example biological components that may be detected/measured include sugar levels, pH levels, CO2 levels (CO2 partial pressure, bicarbonate levels), oxygen levels (oxygen partial pressure, oxygen saturation), temperature, and other such substrates and physiological parameters. The one or more sensors may be configured to sense the presence, absence, or levels of biological components such as, for example, immune system-related molecules (e.g., macrophages, lymphocytes, T cells, natural killer cells, monocytes, other white blood cells, etc.), inflammatory markers (e.g., C-reactive protein, procalcitonin, amyloid A, cytokines, alpha-1-acid glycoprotein, ceruloplasmin, hepcidin, haptoglobin, etc.), platelets, hemoglobin, ammonia, creatinine, bilirubin, homocysteine, albumin, lactate, pyruvate, ketone bodies, ion and/or nutrient levels (e.g., glucose, urea, chloride, sodium, potassium, calcium, iron/ferritin, copper, zinc, magnesium, vitamins, etc.), hormones (e.g., estradiol, follicle-stimulating hormone, aldosterone, progesterone, luteinizing hormone, testosterone, thyroxine, thyrotropin, parathyroid hormone, insulin, glucagon, cortisol, prolactin, etc.), enzymes (e.g., amylase, lactate dehydrogenase, lipase, creatine kinase), lipids (e.g., triglycerides, HDL cholesterol, LDL cholesterol), tumor markers (e.g., alpha fetoprotein, beta human chorionic gonadotrophin, carcinoembryonic antigen, prostate specific antigen, calcitonin), and/or toxins (e.g., lead, ethanol).

It is noted that, while generally referred to as a “sensor” in discussing various embodiments throughout, such “sensors” or sensor components (e.g., sensor 108) may comprise or otherwise be associated with transducers or other components and may be configured as input devices, output devices, or both. For example, such sensors may include ultrasound transducers, optical emitters (e.g., light emitting diodes (LEDs)), optical sensors (e.g., photo diodes), piezoelectric sensors, capacitive sensors, magnetic sensors, and the like.

In one embodiment, a plurality of sensors 108 is spaced along a longitudinally extending portion of the intraluminal device 102. For example, one embodiment may include several pressure sensors that are longitudinally spaced from each other by a distance of approximately 1 centimeter (cm). In one particular embodiment, the intraluminal device may include eleven pressure sensors 108 spaced out over a length of approximately 10 cm along a distal section of the device 102. In other embodiments, other quantities of sensors may be used and different spacings may be employed.

When the intraluminal device 102 is configured as a guidewire, it may include other features and components, such as a coil and/or atraumatic tip formed at the distal end 122. The coil may be a single coil or multiple connected or interwoven coils. Additionally, or alternatively, a polymer material may be positioned on or applied to the distal section of the intraluminal device 102. An atraumatic tip may include a sphere or other curved geometry to protect against potential trauma that might otherwise be caused by the distal end of the intraluminal device 102. The atraumatic tip may be formed from a polymer adhesive material and/or solder, for example.

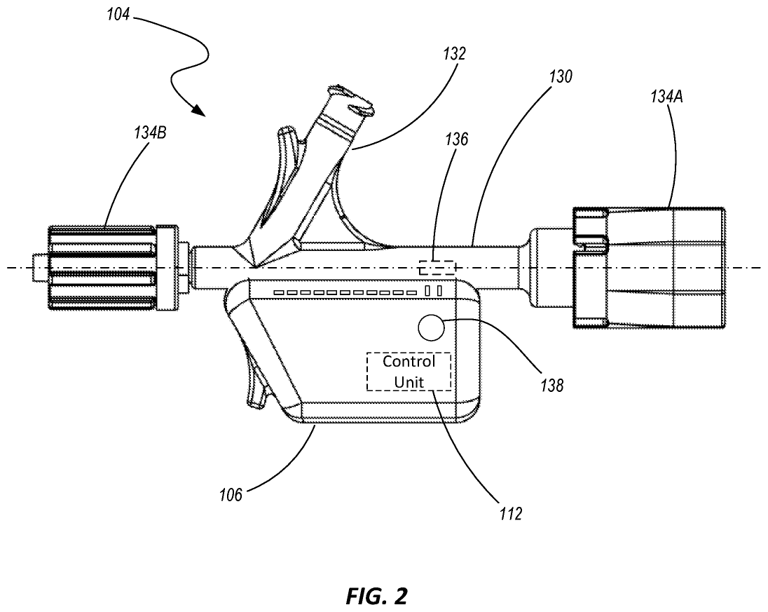

Referring now to FIGS. 2-4, the hemostasis valve 104 and data/power transfer device 106 are described in greater detail in accordance with an embodiment of the present disclosure. The hemostasis valve 104 may include a body portion 130 configured to receive an elongated device, such as a guide wire or catheter (e.g., intraluminal device 102), that can move within the body portion 130 in a direction parallel to its longitudinal axis. The body portion 130 may also include one or more ports 132 having openings that are in fluid communication with the interior of the body portion 130. Connectors 134A and 134B may be located at the proximal and distal ends of the body portion 130, respectively, enabling the valve body 130 to be fluidically coupled with other devices or components during a medical procedure. In one embodiment, the proximal end connector 134A may comprise a conventional connector having threads, a hose barb, or other mechanical connecting structures. In some embodiments, the proximal end connector 134A may be configured with a plunger mechanism configured to open and close an associated valve by displacing the connector 134A in a distal direction. For example, in one non-limiting embodiment, the hemostasis valve 104 may be configured similar to a COPILOT valve available from Abbott Laboratories. In one embodiment, the distal end connector 134B may include a standard luer lock for connecting the valve body 130 to a catheter (e.g., a guide catheter) or other components used in diagnostic, therapeutic and/or interventional medical procedures. One or more valves may be associated with the valve body to prevent fluid form passing out of the valve body 130 even while an intraluminal device is displaced through the body 130 as will be appreciated by those having ordinary skill in the art. The body 130, associated ports 132, and connectors 134A and 134B are not limited to those shown, but may exhibit a variety of configurations or implementations as will be recognized by one having ordinary skill in the art.

The hemostasis valve 104 may include one or more sensors 136 associated with the body 130. For example, the sensor 136 may include a pressure sensor to measure the pressure of the blood within the interior of the valve body 130, which may serve as the aortic pressure (Pa) or “proximal pressure” used in diagnosis and treatment of a stenosis within a blood vessel (e.g., when used in calculation of ratios such as fractional flow reserve (FFR), non-hyperemic pressure ratios (NHPR), other ratios and/or or other diagnosis and treatment techniques). The sensor(s) 136 may be configured for communication with the control unit 112 of the electronic device 106 as described in further detail below. The sensor 136 may be configured as a different type of sensor, such as those described hereinabove, to determine other physiological parameters or biological characteristics of blood present within the hemostasis valve 104.

The hemostasis valve 104 may also include a conductive member (not shown), such as a ring or a plate, configured to serve as a capacitive or other electrical member that facilitates wireless, or non-contact transmission, of power and/or data signals from the electronic device 106 to the intraluminal device 102 via the capacitive member such as described in the various documents previously incorporated by reference (e.g., US20210290100).

As noted above, the electronic device 106 may include a control unit 112 having various components. The electronic device 106 may include one or more input devices, such as a button 138, that may be used to turn the electronic device on or off, to provide other instructions to the control unit (e.g., such as by holding the button for a predetermined amount of time, consecutively pressing the button a predetermined number of times, etc.), or both. While shown generally as a push button, the one or more input devices may additionally or alternatively include other types of switches, sliders, touch sensitive trackpads, touch sensitive input screens, joysticks, directional pads, and the like. In the embodiment shown, the button 138 is positioned on a lateral side of the housing or body of the electronic device 106. However, the button 138 (or other input devices) may be positioned at other locations. For example, the button 138 may be placed on the proximal-most (or back) surface, on the bottom surface (furthest away from the intraluminal device 102), or at some other location.

The electronic device 106 may also include one or more output devices (in addition to the transceiver 118) to provide feedback to a user as will be discussed in further detail below. As discussed below, such an output device may include a display (e.g., an LCD, LED, e-ink, or other greyscale or color display), lights, a speaker, haptic feedback elements, or other appropriate output devices.

The electronic device 106 may additionally include one or more physical features for ergonomic handling of the device 106 in any of its “attached” states and/or when it is detached from the valve 104 and intraluminal device 102. For example, a curved abutment surface or finger grip 139 may be formed on the distal-most surface (or at another location) of the housing or body of the electronic device 106. Such a surface may enable more sure gripping and manipulation of the electronic device 106, for example, when attached to the hemostasis valve 104 and being manipulated by a user, or when being held while the intraluminal device 102 is being displaced relative to the hemostasis valve 104 or otherwise manipulated by a user.

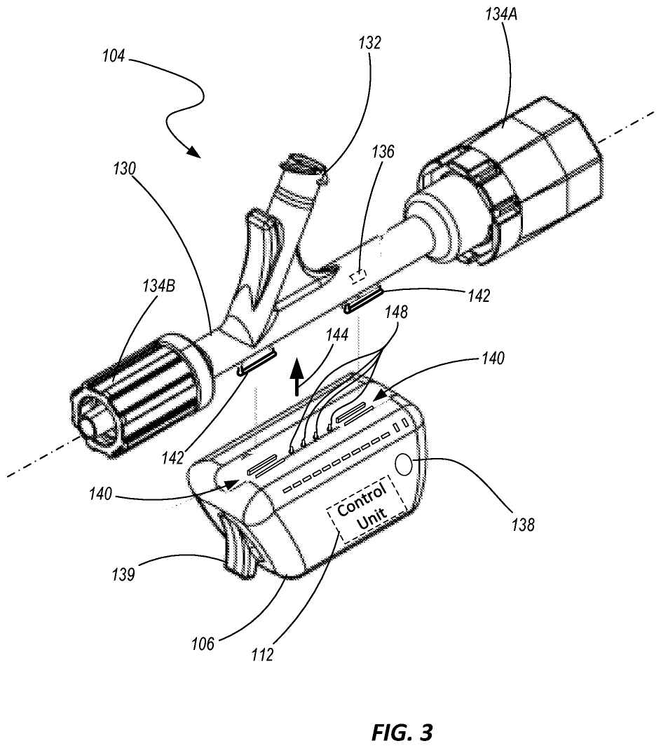

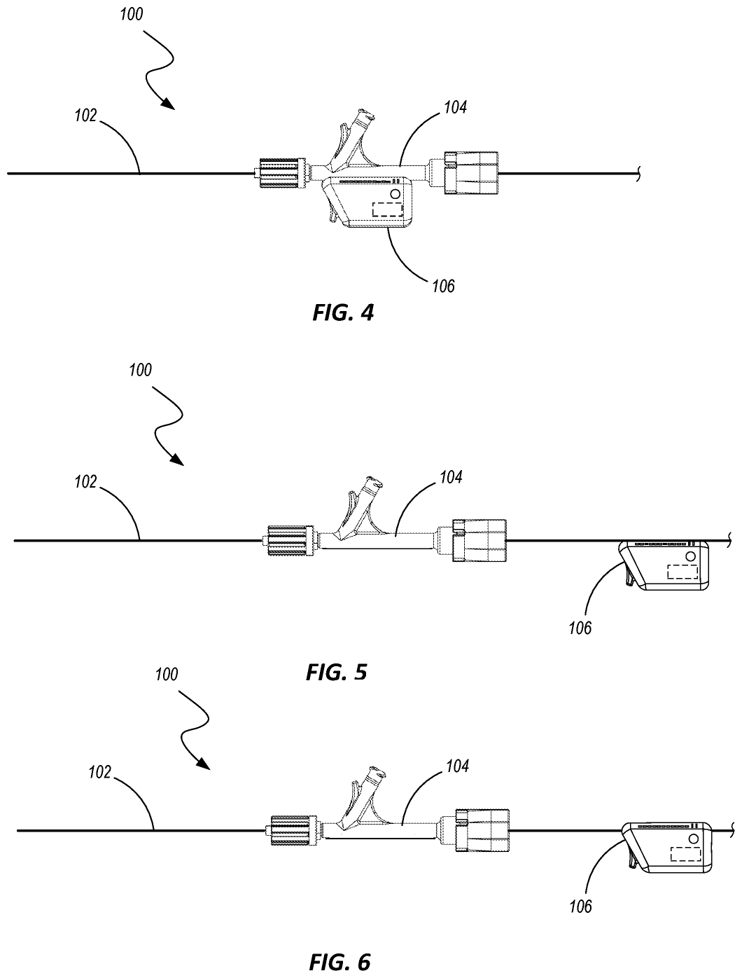

Referring more specifically to FIG. 3, the electronic device 106 may be configured to be removably attached from the hemostasis valve 104. For example, clips 140 or other locking or attachment features may be formed on the electronic device 106 for coupling with associated structures 142 formed on the hemostasis valve 104 (e.g., on the valve body 130). For example, the clips 140 may include “fingers” or other structures that elastically deform to slide over a portion of the mating structures 142 and then engage with a groove or other portion of the mating structures to maintain the electronic device in a position such as depicted in FIGS. 2 and 4. Thus, the electronic device 106 may be coupled with the hemostasis valve 104 by generally aligning the clips 140 and the mating structures 142 and then pressing the electronic device 106 toward the valve body 130 in a direction indicated by arrow 144 until the clips 140 engage and lock with the mating structures 142, holding the electronic device in place relative to the hemostasis valve 104. Removal of the electronic device 106 from the hemostasis valve 104 may be accomplished by applying a force to the electronic device in a direction opposite to that indicated by arrow 144. Of course, other attaching and locking mechanisms may be used to selectively attach or separate the electronic device 106 and the hemostasis valve 104.

As seen in FIG. 3, the electronic device 106 may include one or more electrical contacts 148 configured to contact and electrically communicate with mating contacts (not explicitly shown) formed in, or otherwise associated with, the hemostasis valve 104. The electrical contacts 148 and mating contacts of the hemostasis valve 104 enable the electronic device to electrically communicate with any electrical components associated with the hemostasis valve 104, such as the sensor 136, as well as electrically communicate with the intraluminal member 102, such as by way of an electrical plate (e.g., capacitive plate) positioned in the hemostasis valve as previously discussed. In some embodiments, the electrical contacts 148 may be configured as pogo pins and the mating contacts (on the hemostasis valve 104) may be configured as contact pads (or vice versa). However, other types of electrical connections may also be used, including various contact and/or non-contact mechanisms.

In some embodiments, a sensor (e.g., a pressure sensor) may be associated with, but not necessarily integrated into, the body 130 of the electronic device 106. For example, a pressure sensor may be placed in fluid communication with the interior of the body 130 of the hemostasis valve 104 such as by attaching it to the distal connector 134A. In another embodiment, the sensor may be placed in, or be directly coupled to, one of the available ports 132 of the body 130. In another embodiment, a sensor may be placed in communication with one of the available ports 132 of the body 130 (or with the distal connector 134A) by way of a conduit. In other embodiments, a sensor may be associated with a structure that pierces or punctures through a wall of the body 130 or connects with some other preformed passage to place the sensor in fluid communication with the interior of the body 130. Thus, the electronic device 106 may be used with a hemostasis valve 104 that doesn't include an integrated pressure sensor in some embodiments.

Referring briefly to FIGS. 4-6, various configurations are shown with the electronic device 106 being in electrical communication with the intraluminal device 102. As seen in FIG. 4, the electronic device 106 may be used when connected to a hemostasis valve 104 as has been described above with respect to FIGS. 2 and 3. In some embodiments, the electronic device may additionally be configured for direct connection with the intraluminal device 102, rather than through the hemostasis valve 104. For example, as shown in FIG. 5, the electronic device 106 may be physically coupled with the intraluminal device 102, the intraluminal device being positioned along a side of the body or housing of the electronic device 106. In one embodiment, such coupling may be achieved by a mechanical or structural arrangement (e.g., such as by clips 140 or other structures engaging the intraluminal device 102), it may be accomplished by magnetic forces (when the intraluminal device has known magnetic properties), or it may be accomplished by a combination of both mechanical and magnetic arrangements.

When attached to the intraluminal device 102, electrical contacts (which may be the same as, similar to, different than, or separate from the electrical contacts 148 described above) engage with the intraluminal device 102 to enable transmission of electronic signals (data, power, or both) between the electronic device 106 and sensors 108 or other electrical components of the intraluminal device 102. Thus, in one embodiment, the electronic device 106 is configured for selectively coupling with the hemostasis valve 104 or for direct coupling with the intraluminal device 102 (which may include ohmic coupling or coupling via an electrical field) depending on a variety of factors including a user's preference and the availability of a hemostasis valve configured for electrical and mechanical connection with the electronic device 106. The selectively attachable nature of the electronic device with either the hemostasis valve 104 or the intraluminal device 102 also enables the selection of, and interchangeability of, electronic devices 106 having different components or features (e.g., differing levels of power, differing communication protocols in the transceiver, differing form factors for ergonomic preferences, etc.)

Referring to FIG. 6, in another embodiment, the electronic device 106 may be physically coupled with the intraluminal device 102 by passing the intraluminal device 102 through a channel or elongated passage formed in a body or housing of the electronic device 106 (rather than attaching to a side of the electronic device 106 as shown in FIG. 5). Magnetic and/or mechanical components may still be utilized to more completely secure the intraluminal device 102 to the electronic device. As with the embodiment shown in FIG. 5, when coupled with the intraluminal device 102 as shown in FIG. 6, the electronic device 106 makes and electrical connection with the intraluminal device to enable transmission of electronic signals (data, power, or both) between the electronic device 106 and sensors 108 or other electrical components of the intraluminal device 102. In some embodiments associated with those depicted in FIGS. 5 and 6, an alignment feature may be used to align electrical contacts of the electronic device 106 with electrical contacts or connectors of the intraluminal device 102. For example, a groove, an abutment shoulder, or some other physical feature may be formed on the intraluminal device 102 and configured for engagement with a corresponding structure or mechanism of the electronic device 106.

While in some embodiments, electrical communication between the electronic device 106 and sensors 108 or other electronic components of the intraluminal device 102 may be through physical contact of pads, contacts, connectors or the like, in other embodiments the electronic device 106 may communicate with the sensors 108 through a single conductor (e.g., through the body or physical structure of a guidewire) such as described in the previously incorporated publications (see, e.g., US20210290198).

In either of the embodiments shown in FIGS. 5 and 6, coupling of the electronic device 106 with the intraluminal device 102 may be accomplished such that the electronic device 106 is enabled to rotate relative to the intraluminal device 102 (e.g., about the longitudinal axis of the intraluminal device) while still maintaining electrical communication therewith. Considering an embodiment where the intraluminal device is a guidewire, this means that the guidewire may be torqued and rotated within a vessel of a patient while the electronic device 106 remains in a rotational stationary position relative to the guidewire (i.e., the electronic device 106 does not rotate with the guidewire during a procedure). Such a configuration may provide a user of the system greater ease of use and improved control over the positioning and orientation of the guidewire during a procedure, while maintaining electrical contact between the electronic device and the guidewire.

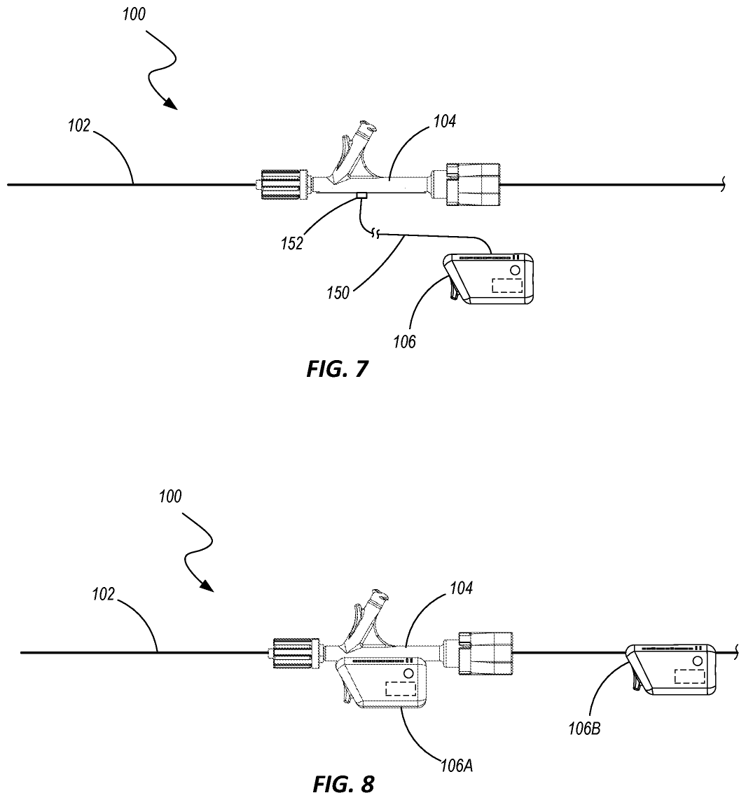

Referring to FIG. 7, in accordance with another embodiment, an electronic device 106 is electronically coupled with the hemostasis valve 104 via a cable 150 and connector 152. The electronic device 106 would function similarly to the embodiment shown and described with respect to FIGS. 3 and 4, except that the electronic device may be physically located remotely away from the hemostasis valve 104.

Referring to FIG. 8, in accordance with another embodiment, multiple electronic devices 106A and 106B may be utilized. For example, one electronic device 106A may be coupled directly with the hemostasis valve 104 such as shown in FIGS. 3 and 4 (or by way of a cable/connector such as shown in FIG. 7), while a second electronic device 106B may be coupled with intraluminal device 102 (such as shown in FIG. 5 or 6).

In one embodiment, the second electronic device 106B may provide power to the sensors 108 or other electronic components of the intraluminal device 102 and may receive data from such components. The first electronic device 106A may provide power to the sensor(s) 136 or other electronic components associated with the hemostasis valve 104 and receive data from such components. The electronic devices 106A and 106B may each transmit and receive information to and from and external device (e.g., device 110), may communicate with each other, or both. For example, the electronic devices 106A and 106B may communicate to each other wirelessly (e.g., through Bluetooth, UWB, Wi-Fi, WiMAX, Zigbee or other such wireless technologies) or through the intraluminal device 102. As such, the electronic devices 106A and 106B may communicate to each other using the same media that they use to communicate and power the sensors 108.

In some embodiments, the electronic devices 106A and 106B may be identical to each other, may be similar to each other, or may be substantially different from one another in form and function (e.g., one primarily providing power while the other primarily receives and relays data). For instance, in some embodiments, the second electronic device 106B may comprise a larger power source and/or may be connected to an outside power source, such that the second electronic device 106B has a greater ability to provide power to the sensor(s) 208 or other electronic components associated with the hemostasis valve 104. In contrast, in some embodiments, the first electronic device 106A may comprise a relatively smaller form than the second electronic device 106B. The first electronic device 106A may be primarily used to receive data from and communicate to the sensor(s) 208 or other electronic components associated with the hemostasis valve 104. Additionally, the first electronic device 106A may be configured to communicate commands to the second electronic device 106B that cause the second electronic device 106B to adjust the power that the second electronic device 106B sends to particular sensors.

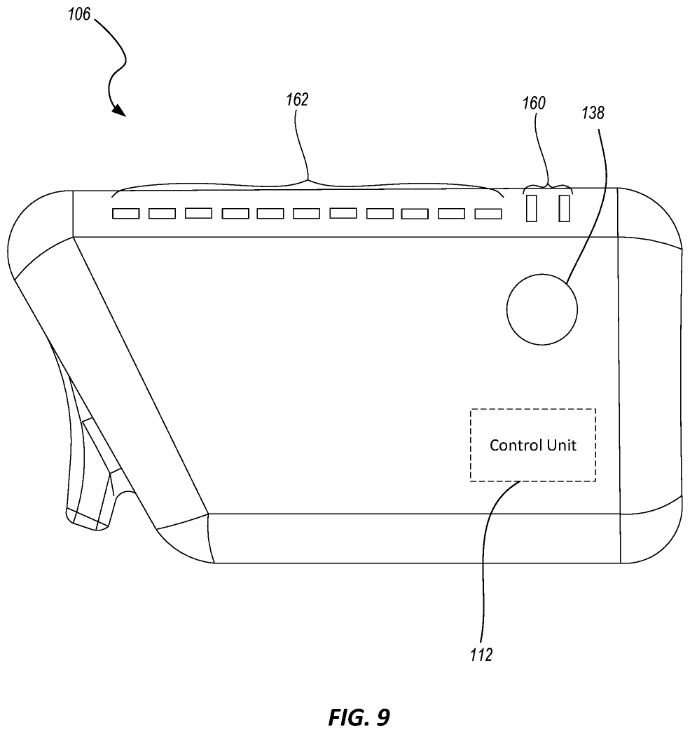

Referring to FIG. 9, an embodiment of the electronic device 106 including output devices 160 and 162 in the form of lights (e.g., LED lights) or other visual indicators. In one embodiment, the output devices 160 may include status indicators (e.g., power on/off, active data transmission, etc.), while output devices 162 may provide indication of data obtained by individual sensors 108 of an intraluminal device (see FIG. 1). In one particular embodiment, the output devices 162 may include an individual light associated with each sensor 108 and may be arranged in a similar pattern to the arrangement of the sensors 108 on the intraluminal device 102 (e.g., the distal-most light representing the distal-most sensor, the proximal-most light representing the proximal-most sensor, and so on). The specific status of a given sensor 108 may be indicated by an associated light of the output device 162. For example, a pressure reading of a given sensor may be indicated by an on/off status, or by a color indicator, of the associated light.

In one particular embodiment, for example, if the system 100 is configured to include a guidewire (as intraluminal device) having a plurality of pressure sensors 108 in order to determine the FFR, NHPR or some other ratio at one or more locations of a vessel, each light of the output device 162 could be “on” or illuminated when the determined ratio at the light's associated sensor is above an identified benchmark, while each light could be “off” or remain in a non-illuminated state if the determined ratio for the associated sensor is below the identified benchmark. In other embodiments, the on/off states could be reversed. In other embodiments, brightness levels or some other parameter may be used for visual indication of the readout or determined parameter of an associated sensor.

In another embodiment, each light of the output device 162 could display a designated color depending on if the associated sensor determines a pressure or a ratio to be within a predefined defined range. For example, in one embodiment, a light may be illuminated in a green color if a ratio (e.g., FFR or NHPR) is determined to be between approximately 0.85 and 1.00 for its associated sensor; a the light may be illuminated as a yellow color if the ratio for the associated sensor is determined to be between approximately 0.80 and approximately 0.85; and the light may be illuminated as a red color if the ratio for the associated sensor is determined to below approximately 0.80. Of course, other ranges may be defined, and other color schemes may be used. Using the individual lights of the output device as indicators of a ratio output derived from the readings of their associated pressure sensors may be a simple and clear indicator for a user of the system 100 to perform (or not perform) an interventional procedure (e.g., balloon, catheter, etc.), without the need for expensive or complicated equipment such as various imaging equipment and monitors such as those currently used in various procedures. Alternatively, such an output may be used as a simple check to verify the information being provided from other equipment such as imaging devices, monitors and co-registration systems.

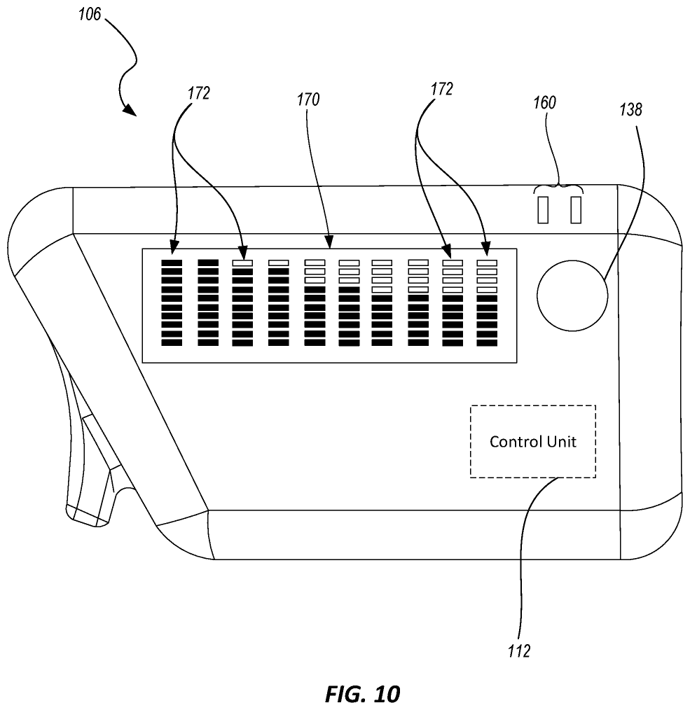

Referring briefly to FIG. 10, an output device 170 associated with an electronic device 106 is shown in accordance with another embodiment. The output device 170 may include a display screen having a plurality of visual indicators 172 (shown as bar graphs) associated with each sensor 108 of the intraluminal device 102. As previously noted, the display of the output device 170 may include an LCD display, an LED display, an e-ink display, or any other appropriate display device. In one embodiment, the visual indicators 172 may represent an approximate ratio (e.g., FFR or NHPR) for an associated sensor 108 based on that sensor's reading as compared to an aortic pressure. For example, each visual indicator may include ten “bars” of a bar graph, with each bar representing 10% of a determined ratio, the ratio being expressed as a range of 0.0 to 1.0. Thus, considering the left-most visual indicator 162, with each of the “bars” being filled or darkened, the visual indicator provides a determination of a ratio that is 1.0. On the other hand, the right-most visual indicator 172 having only six of the “bars” being filled or darkened indicates a determination of a ratio that is 0.6. A user of the system may compare these readings against a benchmark or known standard to determine whether any interventional procedure is warranted. For example, if a ratio of 0.8 or greater is indicated, the user may determine that no interventional procedure is required, while a ratio of less than 0.8 may indicate that some procedure is warranted. As with the embodiment described with respect to FIG. 9, this provides a simplified way of determining or verifying the health of a patient's vessel. Additionally, the visual indicators may help in calibrating or preconditioning the sensors 108.

It is noted that the use of various percentages and ratios are merely examples and that other embodiments may include or use different quantities. It is additionally noted that information could be provided to a display unit in a different format, including different symbols, a reading of actual percentages or ratios, or using other appropriate indicators.

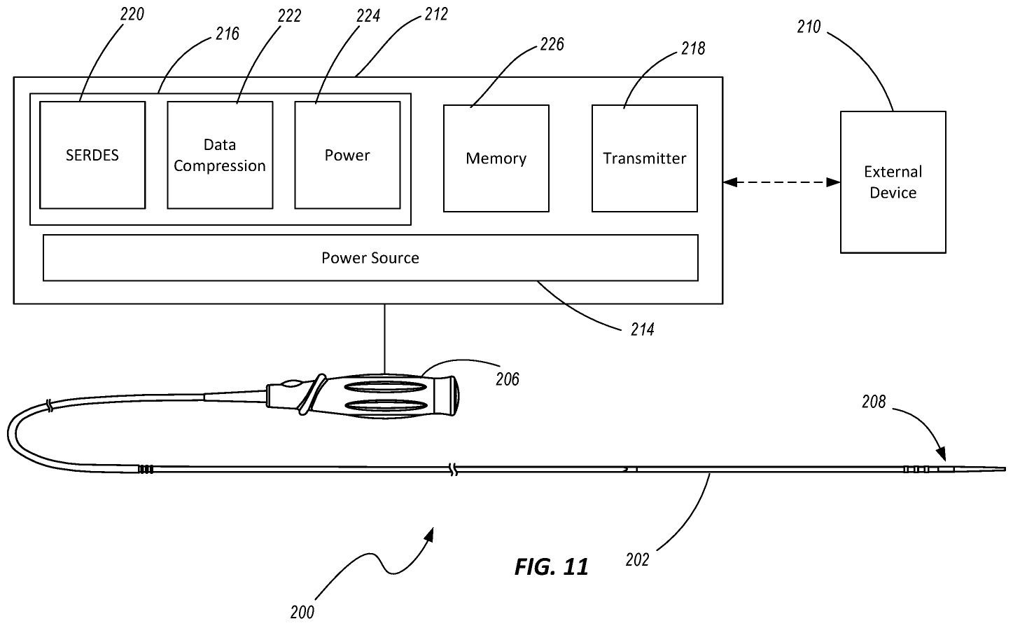

Referring now to FIG. 11, a medical device system 200 is illustrated according to another embodiment of the present disclosure. As shown, the system 200 includes an intraluminal device 202 which may be configured as an elongated member (e.g., a needle, a guidewire, or a catheter) and a data/power transfer device 206 (which may be referred to herein as an electronic device for sake of simplicity) positioned at a proximal end of the intraluminal member 202. One or more sensors 208 may be positioned at a distal end of the intraluminal member 202 and placed in communication with the electronic device 206. The electronic device 206 may include a control unit 212 (shown enlarged and in schematic form) that includes a power source 214 such as a battery, a processor 216, and a transceiver 218. The electronic device 206 may be in communication with an external device 210 (e.g., a stationary or handheld display, tablet computer, or other input and/or output device). In one embodiment, the data transfer device 206 is in wireless communication with the external device 210 (e.g., via the transceiver 218). For example, the transceiver may include components for transmitting and receiving data using Bluetooth, UWB, Wi-Fi, WiMAX, Zigbee or other such wireless technologies. In one embodiment, the processor 216 may include various processing elements, such as a SERDES (serializer/deserializer) block 220 and a data compression block 222 for processing data received from the sensors 208. The processor 216 may also include a power block 224 configured to manage the power transmitted to the sensors 208 and associated circuitry. The control unit 212 may also include memory 226 for storage of data and/or processing instructions.

While the electronic device 206 may be configured to be movable relative to the intraluminal device 202 (such as described in one more embodiments above), in one embodiment, the electronic device 206 may be configured as a handle and be integrated with and/or fixed to the intraluminal device 202 such that a force applied to the electronic device 206 (e.g., axial forces and/or rotational forces relative to the longitudinal axis of the intraluminal device) are transmitted to the intraluminal device 202. Thus, the electronic device 206 may be configured to be ergonomically grasped by a user's hand or fingers and manipulated for the sake of displacing, rotating or otherwise positioning the intraluminal device within a patient's body. It is noted that the electronic device 206 may include a variety of features and components such as those described with respect to other embodiments, including various input and output devices.

In one embodiment, intraluminal device 202 may be configured as a catheter and the sensors 208 of the intraluminal device may include one or more arrays of ultrasonic transducers (e.g., providing an intravenous ultrasound (IVUS) catheter). One or more processors (e.g., application specific integrated circuits (ASICs)) may also be positioned at a distal end of the intraluminal device 202 in association with such ultrasonic transducers. In one embodiment, such transducers may be configured as CMUTs (capacitive micromachined ultrasonic transducers). In one particular embodiment, such transducers may be configured as CMUT on CMOS (complementary metal-oxide-semiconductor) devices. In one embodiment, the arrays may include a plurality of separate arrays, with each array being configured to operate at a specified frequency. In such an embodiment, at least two of the arrays may be configured to operate at frequencies that are different from each other. For example, the arrays may each operate at a different frequency selected from a specified range of frequencies (e.g., from approximately 4 MHz to approximately 50 MHz, although other ranges are also contemplated).

When configured as an IVUS catheter, the processor 214 of the system 200, including its various blocks and processing components, enables the processing and wireless transmission of substantial amounts of imaging data provided by the ultrasound arrays.

In one embodiment, the system 200 may be used in conjunction with a hemostasis valve, with the intraluminal device 202 passing through a body of the hemostasis device, similar to other embodiments described above. In one embodiment, the system 200 may be used in conjunction with the system 100 previously described. For example, the system 200 depicted in FIG. 11 may be configured as an IVUS catheter which may be used with the system 100 shown in FIG. 1 when that system is configured as a pressure sensing guidewire. Additionally, or alternatively, the system 200 may be used with a hemostasis valve 104 that includes a sensor 136 (e.g., a pressure sensor) or other electronic components. In such embodiments, the electronic devices (e.g., 106 and 206) may communicate individually with an external device (e.g., 110), with each other, or both.

One of skill in the art will appreciate that various functions and methods described above may be performed in whole or in part by computer instructions (e.g., software) executed by one or more computer processors. In some embodiments, the one or more processors and/or the one or more computer executable instructions may be stored and/or executed remotely (e.g., in the cloud). As such, the disclosed functions and methods may be executed locally, remotely, or through a distributed computing system that may include the processor 216 within the one or more electronic devices.

The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Headings, lists, and numbering included herein are for ease of explanation only and are not meant to be limiting.

While embodiments of the disclosure may be susceptible to various modifications and alternative forms, specific embodiments have been shown by way of example in the drawings and have been described in detail herein. However, it should be understood that the claimed invention is not intended to be limited to the particular forms disclosed. Rather, the claimed invention includes all modifications, equivalents, and alternatives falling within the spirit and scope of the invention as defined by the following appended claims.

What is claimed is:

1. A medical device system comprising:
a hemostasis valve having a body defining a passage and having at least one electronic component;
an elongated, flexible member configured to be displaced through the passage of the hemostasis valve, the elongated, flexible member including at least one sensor; and
an electronic device removably coupled with the hemostasis valve and being in electrical communication with at least one of the at least one electronic component and the at least one sensor when coupled with the hemostasis valve.

2. The medical device system of claim 1, wherein the at least one sensor includes at least one of pressure sensor, a temperature sensor, a flow sensor, and an imaging sensor.

3. The medical device system of claim 1, wherein the elongated, flexible member includes a guidewire or a catheter.

4. The medical device system of claim 1, wherein the electronic device includes a control unit having a processor, a power source, and a transmitting/receiving device.

5. The medical device system of claim 1, wherein the at least one electronic component includes at least one other sensor.

6. The medical device system of claim 5, wherein the at least one other sensor comprises a pressure sensor.

7. The medical device system of claim 1, wherein the electronic device is configured to communicate wirelessly with an external device.

8. A medical device comprising:
a body defining a passage therethrough;
a first connector at a first end of the body;
a valve at a second end of the body;
the body being configured to receive an elongated, flexible member through the valve and through the passage; and
at least one sensor configured to measure a parameter of a fluid contained within the body.

9. The medical device of claim 8, wherein the at least one sensor includes a pressure sensor.

10. The medical device of claim 9, wherein the body is configured for physical and electrical coupling with an electronic device.

11. The medical device of claim 8, wherein the first connector is configured as a Luer lock.

12. The medical device of claim 8, further comprising at least one port in fluid communication with the passage.

13. An electronic device comprising:
a control unit comprising a body, the control unit including a processor, a device for wirelessly transmitting and receiving data, and a power source disposed in a housing;
wherein the body is configured for selectively coupling with, and removal from, a hemostasis valve.

14. The electronic device of claim 13, further comprising at least one input device and at least one output device.

15. The electronic device of claim 14, wherein the at least one output device includes at least one light.

16. The electronic device of claim 14, wherein the at least one output device includes a plurality of lights, each light of the plurality configured to provide a status indication for an associated sensor of a medical device system.

17. The electronic device of claim 14, wherein the at least one output device is configured to provide at least two status indicators.

18. The electronic device of claim 17, wherein the at least two status indicators include at least two different colors.

19. The electronic device of claim 14, wherein the at least one output device includes a display screen.

20. The electronic device of claim 13, wherein the body is configured for selectively coupling with, and removal from, a flexible, elongated member configured for insertion into a lumen of a patient.
